(19) United States
(12) Patent Application Publication
(10) Pub. No.: US 2024/0221155 A1
(43) Pub. Date: Jul. 4, 2024

(54) EYE IMAGE CAPTURING AND PROCESSING DEVICE

(71) Applicants: NATIONAL TAIPEI UNIVERSITY OF TECHNOLOGY, Taipei City (TW); TAIPEI MEDICAL UNIVERSITY, Taipei City (TW)

(72) Inventors: CHENG-CHUN CHANG, Taipei City (TW); Po-Wen Lu, Taipei City (TW)

(21) Appl. No.: 18/398,320

(22) Filed: Dec. 28, 2023

(30) Foreign Application Priority Data
Dec. 29, 2022 (TW) 111150653

(51) Int. Cl. G06T 7/00; G06T 5/50; G06T 11/00; G06V 10/141; G06V 10/56; G06V 40/18; G16H 50/20

(52) U.S. Cl. CPC G06T 7/0012; G06T 5/50; G06T 11/001; G06V 10/141; G06V 10/56; G06V 40/193; G16H 50/20; G06T 2207/20224; G06T 2207/30041

(57) ABSTRACT

An eye image capturing and processing device is provided. The device includes a portable user terminal body and an application program installed in the portable user terminal body. The portable user terminal body has thereon an image capturing module for capturing an eye image of a user to generate instant image data. The application program receives the instant image data and performs a data processing procedure, and takes an automatic selfie or generates an indication signal to instruct the user to take a manual selfie when a preset condition is satisfied, thereby generating a to-be-diagnosed image corresponding to the eye of the user.

### FIELD OF THE INVENTION

The present disclosure relates to an eye image capturing and processing device, and particularly to a scleral or a conjunctival image capturing and processing device applied to a telehealth system.

### BACKGROUND OF THE INVENTION

Anemia is a major public health issue, seriously affecting human health. Anemia is defined as decreased number of red blood cells or lowered hemoglobin (Hb) concentration in the circulation of blood. It represents that the blood has a lowered ability of carrying oxygen. In a normal condition, if the hemoglobin concentration is not too low, there is no obvious symptom because physiological compensation may work, e.g. increasing cardiac output. When the condition is further aggravated and the physiological compensation can not provide enough oxygen, the patient may has the symptoms such as tiredness, pale skin, irritability, high heart rate, insomnia and headache. Severe anemia will damage cells and vital organs, and even be life-threatening. The clinical standard for diagnosing anemia mainly relies on drawing blood to monitor the hemoglobin concentration in the blood. However, these values fluctuate widely from day to day, and frequent blood collection will cause discomfort to the patients. Further, considerable cost is predictable.

To improve the observation means, many studies have discussed the relation between the anemia and the pallor level in areas of the body. Predicting the anemia condition by observing the pallor level of the eyelid conjunctiva has been commonly used in many areas. This method is even the main criterion for identifying anemia in certain areas where blood draw for measuring hemoglobin concentration is not an easy action. Some literature shows that the anemia detection based on conjunctiva pallor level has high accuracy. Further, jaundice, also known as hyperbilirubinemia, is a yellowish pigmentation of body tissues caused by accumulation of excess bilirubin. Jaundice is typically a sign indicating the liver dysfunction in a clinical diagnosis. In general, the clinical manifestation of jaundice is a yellowish discoloration of the sclera (the white of the eye) or skin. After further clinically considering the patient's medical history involving, for example, use of medications, herbs, dietary supplements and recreational drugs, use of alcohol, risk factors for hepatitis carriers, history of abdominal surgery (especially gallbladder surgery), history of genetic disorder (e.g. liver disease and hemolytic disease), human immunodeficiency virus (HIV) activity and toxic exposures, the clinicians can identify and treat the patients as early as possible. Therefore, detecting jaundice through the color change of the sclera (the white of the eye) is an important chain of disease early warning.

### SUMMARY OF THE INVENTION

An aspect of the present disclosure provides an eye image capturing and processing device. The device includes a portable user terminal body and an application program installed in the portable user terminal body. The portable user terminal body has thereon an image capturing module which captures an eye image of a user to generate instant image data. The application program receives the instant image data and performs a data processing procedure, and takes an automatic selfie or generates an indication signal to instruct the user to take a manual selfie when a preset condition is satisfied, thereby generating a to-be-diagnosed image corresponding to the eye of the user.

In an embodiment, the application program includes a color correction algorithm for transforming an original image obtained through the selfie into the to-be-diagnosed image with true color.

In an embodiment, the eye image capturing and processing device further includes a spectrum sensor chip, in communication with the portable user terminal body, for collecting spectrum distribution of ambient light. The color correction algorithm is a method for spectrally eliminating ambient interference, including steps of: receiving the original image obtained through the selfie; receiving the spectrum distribution collected by the spectrum sensor chip; and adjusting the original image based on the spectrum distribution to eliminate color bias of the original image captured in the ambient light, thereby transforming the original image into a true-color to-be-diagnosed image.

In an embodiment, the eye image capturing and processing device further includes a light source, in communication with the portable user terminal body, for selectively emitting light towards the eye of the user. The spectrum sensor chip or the image capturing module collects the spectrum distribution at least in a first mode and a second mode. In the first mode, the spectrum distribution is collected to obtain first image information when the light source emits the light towards the eye of the user. In the second mode, the spectrum distribution is collected to obtain second image information when the light source does not emit the light. The original image is adjusted to eliminate color bias interference so as to transform the original image into the true-color to-be-diagnosed image, wherein the color bias interference is obtained by subtracting the second image information from the first image information.

In an embodiment, the color correction algorithm includes steps of: receiving the original image obtained through the selfie; and adjusting the original image based on standard eye color or iris color to eliminate color bias of the original image captured in ambient light, thereby transforming the original image into a true-color to-be-diagnosed image.

In an embodiment, the application program executes a position detection algorithm, including steps of: receiving the instant image data; determining whether the eyelid conjunctiva and the sclera of the user are correctly positioned in the instant image data; determining whether the user pull down the eyelid properly; and generating the indication signal or taking an automatic selfie when passing the two determination steps.

In an embodiment, the indication signal controls the portable user terminal body to make a sound or vibrate to instruct the user to take the manual selfie.

In an embodiment, the application program includes a guiding method, comprising steps of: receiving the instant image data; outputting a guiding signal according to the positions of the eyelid conjunctiva and the sclera of the user in the instant image data to instruct the user to adjust relative positions between the portable user terminal body and the face of the user and adjust eyelid opening degree; and generating the indication signal or taking the automatic selfie only when the relative positions and the eyelid opening degree are satisfied.

In an embodiment, the guiding signal is a speech signal, a light flash signal, a vibration signal or a screen display signal for guiding the user.

In an embodiment, the eye image capturing and processing device is applied to a telehealth system and further includes a network module transmitting the to-be diagnosed image to the telehealth system to perform a determination procedure. The determination procedure includes steps of: the telehealth system using an AI engine to perform disease symptom identification based on the to-be-diagnosed image; and the telehealth system outputting a warning signal to the network module or a default emergency contact person or organization when a disease status is diagnosed in the disease symptom identification.

In an embodiment, the telehealth system uploads the diagnosed disease status of the user to a cloud storage space. The diagnosed disease status includes abnormal physiological data of the user. The warning signal includes a link allowing access to the cloud storage space for patient information of the user. Disease diagnosis time and contact information are stored in the cloud storage space for later tracking and process optimization.

In an embodiment, the eye image capturing and processing device includes a determination module, in communication with the application program, for performing disease symptom identification based on the to-be-diagnosed image. When a disease status is diagnosed in the disease symptom identification, a warning signal is outputted to the portable user terminal body or a default emergency contact person or organization, the diagnosed disease status is uploaded to a cloud storage space; and the warning signal includes a link allowing access to the cloud storage space for patient information of the user.

In an embodiment, the disease symptom identification checks symptoms of anemia or jaundice, and the diagnosed disease status includes the anemia or the jaundice.

In an embodiment, the image capturing module and a display of the portable user terminal body are arranged on a first surface and a second surface of the portable user terminal body, respectively, and the first surface is opposite to the second surface.

### BRIEF DESCRIPTION OF THE DRAWINGS

The advantages of the present disclosure will become more readily apparent to those ordinarily skilled in the art after reviewing the following detailed description and accompanying drawings, in which:

FIG. 1 is a functional block diagram illustrating an eye image capturing and processing device according to an embodiment of the present disclosure.

FIG. 2 is a flowchart of a position detection algorithm executed by an application program according to an embodiment of the present disclosure.

FIG. 3A is a flowchart of a guiding method executed by the application program of the present disclosure.

FIG. 3B is schematic diagram showing a screen display signal used in another embodiment of the present disclosure.

FIG. 4 is a functional block diagram illustrating an eye image capturing and processing device according to another embodiment of the present disclosure.

FIG. 5 is a functional block diagram illustrating an eye image capturing and processing device according to a further embodiment of the present disclosure.

### DETAILED DESCRIPTION OF PREFERRED EMBODIMENTS

The present disclosure will now be described more specifically with reference to the following embodiments. It is to be noted that the following descriptions of preferred embodiments of this invention are presented herein for purpose of illustration and description only. It is not intended to be exhaustive or to be limited to the precise form disclosed.

In the following description, specific details are given to provide a clear explanation of various aspects of the present disclosure. However, one of ordinary skill in the arts will understand that the present disclosure can be practiced without specifying known details of steps, operations, elements, circuits, components and/or features. For example, commonly used functional circuit modules are represented by blocks in the diagrams without introducing redundant description. In the embodiments, known circuits, structures and technologies are not shown in excessive details in order to simplify the description to be in a concise manner. The embodiments used herein are for the purpose of illustration and are not intended to limit the present disclosure. The features adopted in any embodiment are not construed as superior to other features proposed in the present disclosure.

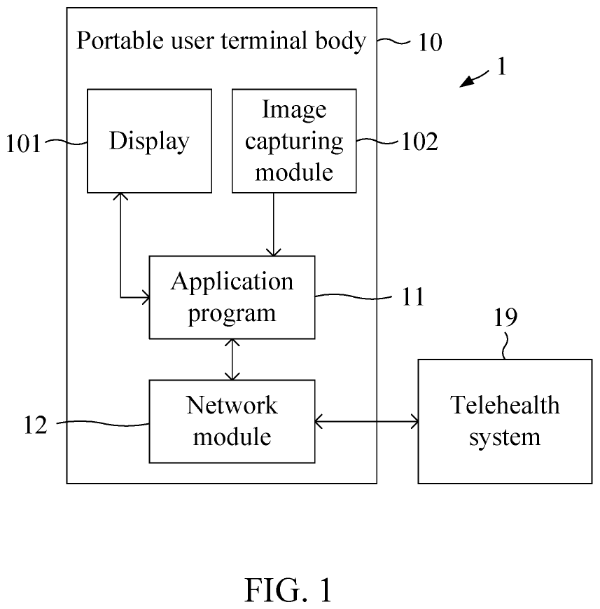

Please refer to FIG. 1, which is a functional block diagram illustrating an eye image capturing and processing device according to an embodiment of the present disclosure. The eye image capturing and processing device 1 is applicable to a telehealth system 19. The eye image capturing and processing device 1 mainly includes a portable user terminal body 10, an application program 11 loaded in the portable user terminal body 10, and a network module 12 disposed in the portable user terminal body 10. The portable user terminal body 10 has thereon a display 101 and an image capturing module 102. The image capturing module 102 is configured to continuously capture target images (e.g. eye images) of a user to generate instant image data. The application program 11 loaded in the portable user terminal body 10 is configured to receive the instant image data and perform a data processing procedure. If a preset condition is satisfied, the application program 11 takes an automatic selfie or generates an indication signal to instruct the user to take a manual selfie to generate a to-be-diagnosed image corresponding to the eye. The network module 12 is disposed in the portable user terminal body 10 and in communication with the application program 11 and the telehealth system 19. The network module 12 is configured to transmit the to-be-diagnosed image to the telehealth system 19 for later symptoms identification procedure.

The following examples are given to make the present disclosure more clearly. The image capturing and processing device 1 can be implemented by a smartphone, a tablet computer or other similar portable information device. A smartphone is adopted in this embodiment. The smartphone has installed application program 11 and the network module 12. The portable user terminal (i.e. the smartphone) body 10 includes the display 101 and the image capturing module 102. The image capturing module 102 is configured to continuously capture target images (e.g. eye images) of a user to generate the instant image data. According to general settings of a smartphone, the primary camera (the image capturing module 102) having better performance is located on the rear of the smartphone, and the display 101 is located on the front of the smartphone. Hence, the image capturing module 102 and the display 101 are arranged on a first surface and a second surface of the portable user terminal body 10, respectively, wherein the first surface and the second surface face opposition directions. When the user holds the smartphone to make the image capturing module 102 face towards the eye, the user can not see the instant images through the display 101 which is located on the other surface of the smartphone. The application program 11 of the present disclosure is developed to perform a method to solve this problem.

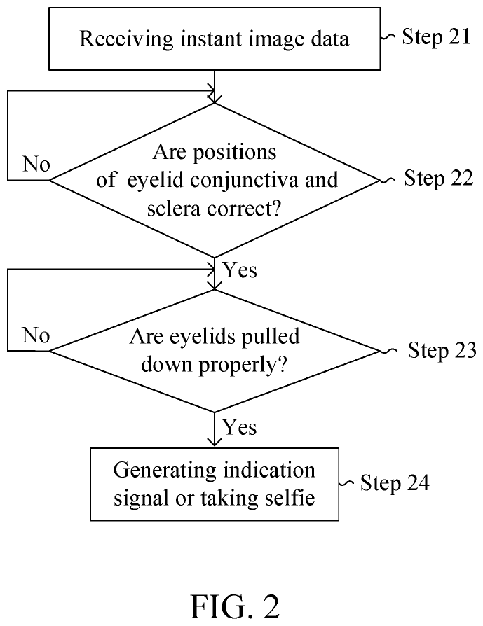

As shown in FIG. 2, the application program 11 in the embodiment performs a position detection algorithm including steps of: receiving the instant image data (Step 21); determining whether positions of the eyelid conjunctiva and the sclera of the user are correct in the instant image data (Step 22); determining whether the user pull down the eyelids properly to expose the eyelid conjunctiva (Step 23); and generating the indication signal or automatically taking a selfie when the two preset conditions are satisfied (Step 24). The indication signal controls the portable user terminal body 10 to make a sound or vibrate to instruct the user to take a selfie. Thus, the to-be-diagnosed image corresponding to the target (i.e. the eye) is generated. Therefore, the to-be-diagnosed image can be correctly obtained through automatic selfie or manual selfie in response to the indication signal when the eyelid conjunctiva(s) and the sclera(s) of the user are located in the correct positions and the eyelid(s) are properly pulled down to expose the eyelid conjunctiva(s). In other words, the lens of the image capturing module 102 points to the eye(s) correctly.

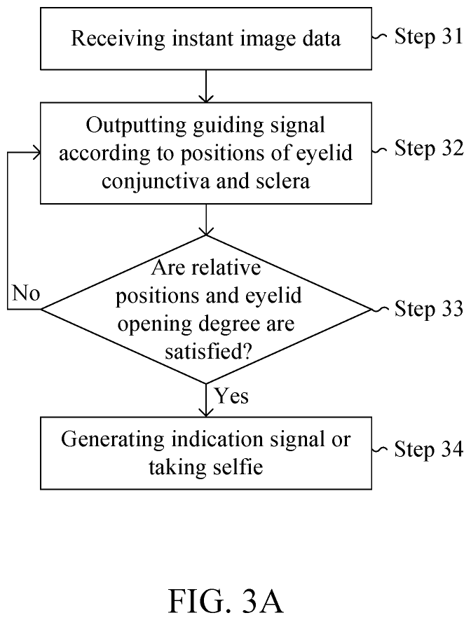

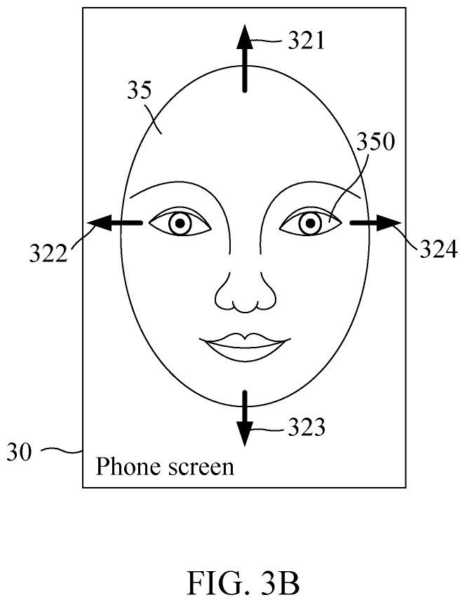

To assist the user in obtaining the correct to-be-diagnosed image, the application program 11 of the present disclosure further performs a guiding method as shown in FIG. 3A, including steps of: receiving the instant image data (Step 31); outputting a guiding signal according to the positions of the eyelid conjunctiva and the sclera of the user in the instant image data (Step 32) to instruct the user to adjust the relative positions between the portable user terminal body 10 and the face of the user and adjust the eyelid opening degree; determining whether the relative positions and the eyelid opening degree are satisfied (Step 33); generating the indication signal or taking the automatic selfie if it is determined YES in Step 33 (Step 34); and otherwise returning Step 32 to repeating the guiding action. If a rear-facing camera (primary camera) is used in the guiding method, the guiding signal may be an artificial speech signal guiding the user to move the phone/camera up, down, left, right, or move the phone/camera closer to or away from the user, etc. Also, the artificial speech signal may guide the user to look up, look down, turn left, turn right, etc. Further, the artificial speech signal may guide the user to increase the eyelid opening degree or pull the lower eyelid further down. In another embodiment, the guiding signal may be a LED flash signal or vibration signal. For example, stronger or faster flash or vibration may represent that the target is closer to the preferred position in some applications. It is also applicable that stronger or faster flash or vibration may represent that the target is father from the preferred position in other applications. This guiding method is helpful for the image capturing and processing device 1 of the present disclosure to capture the image ready for diagnosis.

FIG. 3B is schematic diagram showing that a screen display signal is used in another embodiment of the present disclosure. If a front-facing camera (selfie camera) is used in the guiding method, the user face 35, especially the eye(s) 350, is shown on the phone screen 30. The arrows 321, 322, 323 and 324 are additionally provided on screen to indicate the desired movement direction.

Moreover, in order to enhance the accuracy of disease symptom identification, the application program 11 of the present disclosure may further include a color correction algorithm to transform the original image obtained through the selfie into a true-color or substantially true-color to-be-diagnosed image. Hence, the color correction algorithm may be an automatic white balance operation for eliminating unnatural color casts from ambient light. The color correction algorithm includes steps of: receiving the original image obtained through the selfie; and adjusting the color of the original image based on standard eye color (iris color) to eliminate color bias of the original image captured in the ambient light, thereby transforming the original image into the to-be-diagnosed image with substantially true color. The standard iris colors corresponding to various races could be recorded in a built-in table (not shown) stored in the system. When operating the application program 11, the user may select the corresponding standard iris color from the built-in table. In an embodiment, the race is determined according to the color of the iris or pupil in the original image obtained through the selfie, and then the standard iris color corresponding to the race is used to adjust the original image. For example, dark brown eyes usually correspond to the races in the Africa, East Asia or the Southeast Asia, and light brown eyes usually correspond to the races in West Asia, the Americas or Europe.

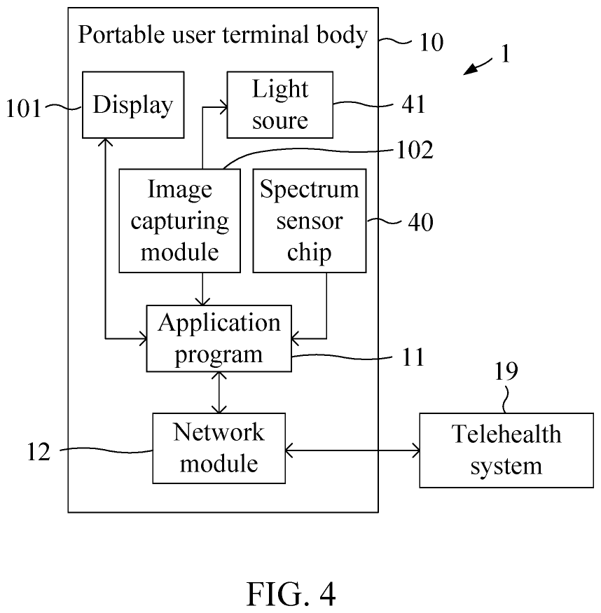

Please refer to FIG. 4, which is a functional block diagram illustrating an eye image capturing and processing device according to another embodiment of the present disclosure. The portable user terminal body 10 is in communication with a spectrum sensor chip 40, configured to collect the spectrum distribution of the ambient light during the image capturing. The spectrum sensor chip 40 may be integrated in the portable user terminal body 10 or located at a separate part from the portable user terminal body 10, e.g. at a periphery near the eyes, and thus the spectrum distribution of the ambient light near the eyes can be collected conveniently. Hence, the color correction algorithm may be a method for spectrally eliminating the ambient interference, including steps of: receiving the original image obtained through the selfie; receiving the spectrum distribution collected by the spectrum sensor chip; and adjusting the color of the original image to eliminate color bias of the original image captured in the ambient light, thereby transforming the original image into the to-be-diagnosed image with substantially true color.

In addition, a light source 41 could be provided and disposed on the portable user terminal body 10. The light source 41 is in communication with the image capturing module 102 of the portable user terminal body 10, and configured to be controlled to be switched on or off to selectively emit light towards the eye(s) of the user. The spectrum sensor chip 40 and/or the image capturing module 102 will collect the spectrum distribution in at least two modes, In the first mode, the light source 41 is switched on to emit light towards the eye(s); and in the second mode, the light source 41 is switched off and no addition lighting is provided. Subtracting the image information of the second mode from the image information of the first mode will obtain the color bias interference due to the light source 41. The original image is adjusted by applying the above-described color correction algorithm and removing the color bias interference-related part to eliminate color bias of the original image, thereby transforming the original image into the to-be-diagnosed image with substantially true color.

The network module 12 transmits the to-be-diagnosed image to the telehealth system 19 for analysis and diagnosis. The related determination procedure includes the following steps. The telehealth system 19 uses an AI engine (not shown) to perform disease symptom identification through the to-be-diagnosed image. When a disease status is diagnosed, e.g. determining that a physiological data obtained from the to-be-diagnosed image does not fall in a normal range and is considered abnormal, the telehealth system 19 outputs a warning signal to the network module 12 or a default emergency contact person or organization. The telehealth system 19 uploads the diagnosed disease status of the user to a cloud storage space, and the warning signal includes a link allowing access to the cloud storage space for the patient information. The disease symptom corresponds to anemia or jaundice, and the diagnosed disease is anemia or jaundice. The cloud storage space also stores the patient information and the abnormal physiological data including the diagnosis result related to anemia or jaundice.

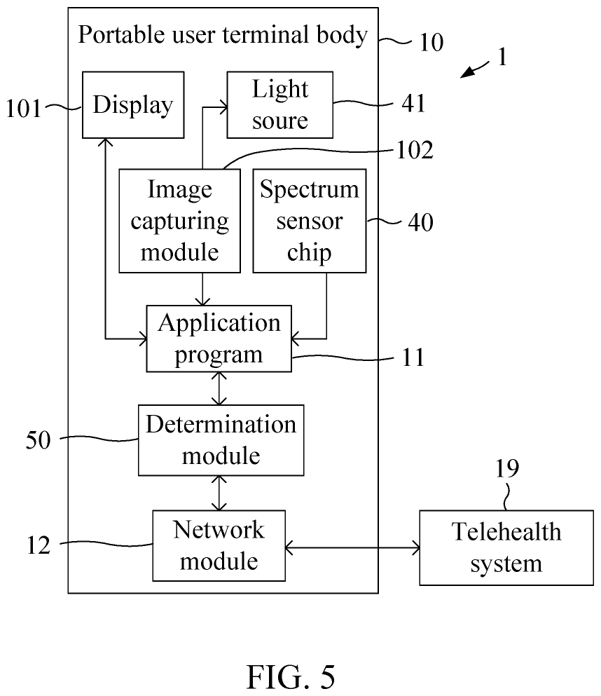

Please refer to FIG. 5, which is a functional block diagram illustrating an eye image capturing and processing device according to a further embodiment of the present disclosure. Instead of the AI engine in the telehealth system 19, the disease symptom identification could be performed by edge computing implemented by local hardware. In this diagram, the eye image capturing and processing device 1 of the present disclosure further includes a determination module 50, in communication with the application program 11. The determination module 50 is configured to share or complete the operation assigned to the AI engine in the above embodiment. The determination module 50 performs the disease symptom identification according to the to-be-diagnosed image. When a disease status is diagnosed, e.g. determining that a physiological data does not fall in a normal range and is considered abnormal, the telehealth system 19 outputs a warning signal to the portable user terminal body 10 or a default emergency contact person (e.g. notifying designated relative's mobile phone via SMS) or organization (e.g. the responsible care center or hospital). Or the telehealth system 19 may upload the diagnosed disease status of the user to a cloud storage space, and the warning signal includes a link allowing access to the cloud storage space for the patient information. The related history such as the disease diagnosis time and the contact information are also stored in the cloud storage space for later tracking and process optimization. Optionally, the network module 12 in the embodiment with reference to FIG. 5 may be omitted. The operation about the disease symptom identification based on the to-be-diagnosed image is performed by the determination module 50 disposed in the portable user terminal body 10 and the related data are stored in the portable user terminal body 10.

According to the concepts of the present disclosure, the eye image can be conveniently and properly captured for diagnosis. Through the portable user terminal body with specific application program, the user can check his or her health status preventively whenever and wherever possible. It is advantageous to public health.

While the disclosure has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An eye image capturing and processing device, comprising:
a portable user terminal body having thereon an image capturing module which captures an eye image of a user to generate instant image data; and
an application program, installed in the portable user terminal body, for receiving the instant image data and performing a data processing procedure, and taking an automatic selfie or generating an indication signal to instruct the user to take a manual selfie when a preset condition is satisfied, thereby generating a to-be-diagnosed image corresponding to the eye of the user.

2. The eye image capturing and processing device according to claim 1, wherein the application program includes a color correction algorithm for transforming an original image obtained through the selfie into the to-be-diagnosed image with true color.

3. The eye image capturing and processing device according to claim 2, further comprising a spectrum sensor chip, in communication with the portable user terminal body, for collecting spectrum distribution of ambient light, the color correction algorithm being a method for spectrally eliminating ambient interference, the method comprising steps of:
receiving the original image obtained through the selfie;
receiving the spectrum distribution collected by the spectrum sensor chip; and
adjusting the original image based on the spectrum distribution to eliminate color bias of the original image captured in the ambient light, thereby transforming the original image into a true-color to-be-diagnosed image.

4. The eye image capturing and processing device according to claim 3, further comprising a light source, in communication with the portable user terminal body, for selectively emitting light towards the eye of the user, and the spectrum sensor chip or the image capturing module collects the spectrum distribution at least in a first mode and a second mode, wherein
in the first mode, the spectrum distribution is collected to obtain first image information when the light source emits the light towards the eye of the user,
in the second mode, the spectrum distribution is collected to obtain second image information when the light source does not emit the light, and
the original image is adjusted to eliminate color bias interference so as to transform the original image into the true-color to-be-diagnosed image, wherein the color bias interference is obtained by subtracting the second image information from the first image information.

5. The eye image capturing and processing device according to claim 2, wherein the color correction algorithm comprises steps of:
receiving the original image obtained through the selfie; and
adjusting the original image based on standard eye color or iris color to eliminate color bias of the original image captured in ambient light, thereby transforming the original image into a true-color to-be-diagnosed image.

6. The eye image capturing and processing device according to claim 1, wherein the application program executes a position detection algorithm, comprising steps of:
receiving the instant image data;
determining whether the eyelid conjunctiva and the sclera of the user are correctly positioned in the instant image data;
determining whether the user pull down the eyelid properly; and
generating the indication signal or taking an automatic selfie when the two determination steps are satisfied.

7. The eye image capturing and processing device according to claim 6, wherein the indication signal controls the portable user terminal body to make a sound or vibrate to instruct the user to take the manual selfie.

8. The eye image capturing and processing device according to claim 6, wherein the application program comprises a guiding method, comprising steps of:
receiving the instant image data;
outputting a guiding signal according to positions of the eyelid conjunctiva and the sclera of the user in the instant image data to instruct the user to adjust relative positions between the portable user terminal body and the face of the user and adjust eyelid opening degree; and
generating the indication signal or taking the automatic selfie only when the relative positions and the eyelid opening degree are satisfied.

9. The eye image capturing and processing device according to claim 8, wherein the guiding signal is a speech signal, a light flash signal, a vibration signal or a screen display signal for guiding the user.

10. The eye image capturing and processing device according to claim 1, wherein the eye image capturing and processing device is applied to a telehealth system and further comprises a network module transmitting the to-be diagnosed image to the telehealth system to perform a determination procedure, the determination procedure comprising steps of:
the telehealth system using an AI engine to perform disease symptom identification based on the to-be-diagnosed image; and
the telehealth system outputting a warning signal to the network module or a default emergency contact person or organization when a disease status is diagnosed in the disease symptom identification.

11. The eye image capturing and processing device according to claim 10, wherein the telehealth system uploads the diagnosed disease status of the user to a cloud storage space wherein the diagnosed disease status comprises abnormal physiological data of the user; the warning signal includes a link allowing access to the cloud storage space for patient information of the user; and disease diagnosis time and contact information are stored in the cloud storage space for later tracking and process optimization.

12. The eye image capturing and processing device according to claim 10, wherein the disease symptom identification checks symptoms of anemia or jaundice, and the diagnosed disease status comprises the anemia or the jaundice.

13. The eye image capturing and processing device according to claim 1, further comprising a determination module, in communication with the application program, for performing disease symptom identification based on the to-be-diagnosed image, wherein when a disease status is diagnosed in the disease symptom identification,
a warning signal is outputted to the portable user terminal body or a default emergency contact person or organization;
the diagnosed disease status is uploaded to a cloud storage space; and
the warning signal includes a link allowing access to the cloud storage space for patient information of the user.

14. The eye image capturing and processing device according to claim 13, wherein the disease symptom identification checks symptoms of anemia or jaundice, and the diagnosed disease status comprises the anemia or the jaundice.

15. The eye image capturing and processing device according to claim 1, wherein the image capturing module and a display of the portable user terminal body are arranged on a first surface and a second surface of the portable user terminal body, respectively, and the first surface is opposite to the second surface.
